(19) United States
(12) Patent Application Publication
(10) Pub. No.: US 2010/0093106 A1
(43) Pub. Date: Apr. 15, 2010

(54) Amine-Reactive Biosensor

(75) Inventors: Krista Leah Witte, Hayward, CA (US); Henrik Persson, Mountain View, CA (US); Sae Choo, San Jose, CA (US); Robert Zuk, Atherton, CA (US)

Correspondence Address: FENWICK & WEST LLP, SILICON VALLEY CENTER, 801 CALIFORNIA STREET, MOUNTAIN VIEW, CA, 94041, US

(73) Assignee: FORTEBIO, INC., Menlo Park, CA (US)

(21) Appl. No.: 12/440,872

(22) PCT Filed: Sep. 14, 2007
(86) PCT No.: PCT/US07/20062

§ 371 Date: Dec. 22, 2009

(30) Foreign Application Priority Data
Sep. 14, 2006 (US) 60825646

(51) Int. Cl. G01N 33/53

(52) U.S. Cl. 436/501

(57) ABSTRACT

Described are methods, articles of manufacture, and kits for coupling an analyte-binding molecule to the surface of a biosensor through formation of amide bonds. One amide bond is formed between a first carboxyl group on a polymer and a first reflecting surface comprising an aminoalkyl moiety. A second amide bond is formed between a second carboxyl group on the polymer and an amine group on an analyte-binding molecule to be coupled. The present invention thus provides for covalent attachment of the analyte-binding molecule to the biosensor, thus providing advantages over non-covalent attachment methods of the prior art. These advantages include the ability to couple without using bio tin (which, in some instances can alter functional properties of a molecule) the ability to improve the fidelity with which a binding or dissociation reaction that takes place on the surface of the biosensor represents a solution phase reaction, and the ability to regenerate the sensor by stripping ligands from the covalently bound analyte-binding molecule.

### CROSS REFERENCE TO RELATED APPLICATIONS

This application claims the benefit of U.S. Provisional Application Ser. No. 60/825,626, filed Sep. 14, 2006, the entire contents of which is incorporated by reference for all purposes.

### STATEMENT REGARDING FEDERALLY SPONSORED RESEARCH OR DEVELOPMENT

Not applicable.

### BACKGROUND OF THE INVENTION

1. Field of the Invention

The present invention relates to methods, articles of manufacture, and kits directed to amine-reactive biosensors.

2. Description of the Related Art

Diagnostic tests based on a binding event between members of an analyte-anti-analyte binding pair are widely used in medical, veterinary, agricultural and research applications. Typically, such methods are employed to detect the presence or amount or an analyte in a sample, and/or the rate of binding of the analyte to the anti-analyte. Typical analyte-anti-analyte pairs include complementary strands of nucleic acids, antigen-antibody pairs, and receptor-receptor binding agent, where the analyte can be either member of the pair, and the anti-analyte molecule, the opposite member.

Diagnostics methods of this type often employ a solid surface having immobilized anti-analyte molecules to which sample analyte molecules will bind specifically and with high affinity at a defined detection zone. In this type of assay, known as a solid-phase assay, the solid surface is exposed to the sample under conditions that promote analyte binding to immobilized anti-analyte molecules. The binding event can be detected directly, e.g., by a change in the mass, reflectivity, thickness, color or other characteristic indicative of a binding event. Where the analyte is pre-labeled, e.g., with a chromophore, or fluorescent or radiolabel, the binding event is detectable by the presence and/or amount of detectable label at the detection zone. Alternatively, the analyte can be labeled after it is bound at the detection zone, e.g., with a secondary, fluorescent-labeled anti-analyte antibody.

Co-owned U.S. Pat. No. 5,804,453 (the '453 patent), which is incorporated herein by reference, discloses a fiber-optic interferometer assay device designed to detect analyte binding to a fiber-optic end surface. Analyte detection is based on a change in the thickness at the end surface of the optical fiber resulting from the binding of analyte molecules to the surface, with greater amount of analyte producing a greater thickness-related change in the interference signal. The change in interference signal is due to a phase shift between light reflected from the end of the fiber and from the binding layer carried on the fiber end, as illustrated particularly in FIGS. 7a and 7b of the '453 patent. The device is simple to operate and provides a rapid assay method for analyte detection.

Co-owned U.S. pre-grant publication number 2005-0254062 (the '062 application), which is incorporated herein by reference, discloses an improved fiber-optic interferometer assay device that includes a spacing of at least 50 nm between a first and a second reflecting surface. That spacing modulates the change in interference signal into the visible range to improve the device performance. The '062 application discloses several methods for coupling an analyte-binding molecule to a biosensor. These include linkages through biotin, and covalent linkage of amine-containing molecules through an NHS-ester (see Example 4). The covalent coupling chemistry disclosed in the '062 application uses a bifunctional linker between the surface chemistry coating on the optical element and the analyte-binding molecule. The '062 chemistry is based on sulfo-SMCC (sulfosuccinimidyl 4-[N-maeleimidomethyl]cyclohexane-1-carboxylate) and requires thiol groups to be on the surface of the optical element. While this provides for covalent bonding between the analyte-binding molecule and the optical element, it can in some instances, result in less than optimal performance and difficulties in manufacture, especially at commercial scales. Thiol functional groups can be unstable and prone to oxidation. In some cases, a less than optimal level of non-specific binding of analyte may occur. In other instances, surface charges present on the glass may create surface potentials that can affect analyte binding or attachment of the analyte-binding molecule to the optical element.

The present invention is designed to overcome the limitations of the prior art by providing an amine-reactive biosensor useful for covalently coupling a layer of analyte-binding molecules to a polycarboxylic acid-containing polymer which is itself coupled to a first reflecting surface. The present invention also contemplates biosensors that include the covalently-coupled analyte-binding molecule layer. Covalent attachment of the analyte-binding molecule is more stable than non-specific adsorption methods of the prior art. The chemistry used in the methods of the present invention is useful for reducing non-specific binding through its incorporation of polycarboxylic acid-containing polymers including polypeptides such as, e.g., bovine serum albumin (BSA). The chemistry also is believed to provide an advantage by improving the screening of surface charges that may be present on a glass or other charged surface to reduce binding artifacts they can create, and in embodiments in which the polycarboxylic acid-containing polymer is a macromolecule (such as, e.g., BSA or other polypeptides), by providing a macromolecular spacer that improves native binding activity of the interaction under study by distancing that interaction from the sensor surface.

The methods of the invention also provide an important advantage in that analyte-binding molecules can be covalently immobilized onto the optical assembly from solutions that later can be recovered and re-used. In certain formats useful for practicing the methods of the invention, a sample of analyte-binding molecule remains in a container such as a multi-well plate, preventing it from being lost to dead volume or diluted with buffers. The present invention also can be adapted to permit batch immobilization of one or more analyte-binding molecules to multiple assemblies in parallel. The present invention uses simple chemistry and eliminates the need for biotinylation, which may inactivate some analyte-binding molecules, and provides for sensors that are sufficiently robust to permit regeneration using a variety of conditions to remove ligands bound to the analyte-binding molecule without stripping the analyte-binding molecule from the sensor.

### SUMMARY OF THE INVENTION

The invention includes, in one aspect, a method for derivatizing an optical assembly comprising an optical element adapted for coupling to a light source via a fiber with a layer of an analyte-binding molecule comprising an amine group to provide a biosensor that can be used in an apparatus for detecting an analyte in a sample, including detecting the presence of an analyte, the amount of analyte or the rate of association and/or dissociation of analyte to an analyte-binding molecule.

The optical element has a proximal reflecting surface and a distal reflecting surface. Once derivatized, the optical element also includes a layer of analyte-binding molecules covalently coupled to the element and positioned so that the interference between beams reflected from the proximal and distal reflecting surfaces varies as analyte binds to the layer of analyte-binding molecules.

In one particular design, the distal reflecting surface includes the layer of analyte-binding molecules. As analyte binds to the layer of analyte-binding molecules, the optical path length or the physical distance between the two reflecting surfaces may increase, for example. In another aspect of the invention, a transparent solid material is located between the reflecting surfaces and, optionally, the proximal reflecting surface includes a material with an index of refraction greater than that of the transparent solid material. Alternately, an air gap may be located between the reflecting surfaces. In yet another design, the distal reflecting surface is positioned between the proximal reflecting surface and the layer of analyte-binding molecules. For example, analyte binding may cause the layer of analyte-binding molecules to swell, moving the distal reflecting surface closer to the proximal reflecting surface. In yet another design, the layer of analyte-binding molecules is positioned between the two reflecting surfaces. Analyte binding may cause the layer to swell or to change its refractive index, thus changing the interference between the two reflected beams.

In another aspect, the optical assembly has first and second reflecting surfaces separated by a distance “d” greater than 50 nm. The optical assembly is composed of a transparent optical element that can have a thickness defined between proximal and distal faces of the element of at least 50 nm, preferably between 400-1,000 nm. The first reflecting surface is carried on the distal face of optical element, and comprises a layer of analyte-binding molecules. The second reflecting surface is formed by a coating of transparent material having an index of refraction greater than that of the optical element. This coating can be formed of a Ta₂O₅ layer having a preferred thickness of between 5 and 50 nm. The optical element can be SiO₂, and has a thickness of between about 100-5,000 nm, preferably 400-1,000 nm.

The methods of the present invention can be practiced using any of these optical assemblies. The methods include activating carboxyl groups on a polymer by exposing the polymer to a water-soluble carbodiimide and an N-hydroxysuccinimide, exposing the activated carboxyl group to a reaction mixture that includes an analyte-binding molecule comprising an amine group, and quenching the reaction, thereby generating a first amide bond between the polymer and the optical assembly and a second amide bond between the polymer and the analyte-binding molecule.

In one aspect, the polymer is a polypeptide, such as bovine serum albumin, BSA. In another aspect, the analyte-binding molecule is a polypeptide or a nucleic acid. In one aspect, the water-soluble carbodiimide is EDC. In yet another aspect, the method includes exposing the optical element to a sugar-containing solution and subsequently drying the optical element.

In still other aspects, the methods of the invention include steps for regenerating an optical assembly that has been derivatized according to any of the methods of the invention and subsequently used to measure binding of an analyte to the analyte-binding molecule. Regeneration is accomplished by exposing the optical assembly to a chaotrope to remove a non-covalently bound analyte from the optical element. In one aspect, the chaotrope is an acid, a base, a salt, a detergent, urea or guanidinium.

In still another aspect, the invention includes optical assemblies produced in accordance with the methods of the invention.

In yet other aspects, the invention includes a kit for derivatizing an optical assembly with a layer of analyte-binding molecules that include an amine group. In some embodiments, the kit comprises at least three of the following components: a polycarboxylated polymer, a water-soluble carbodiimide, an N-hydroxysuccinimide, a quencher, instructions for use, and packaging.

In one aspect, the water-soluble carbodiimide provided in the kit is EDC. In another aspect, the polycarboxylated polymer provided in the kit is a polypeptide.

In another aspect, the kit further comprises a sugar. In yet another aspect the kit further comprises a chaotrope. In one aspect, the chaotrope is an acid, a base, a salt, a detergent, urea, or guanidinium.

In still another aspect of the invention, the kit includes an optical assembly comprising an optical element adapted for coupling to a light source via a fiber, the optical element comprising a transparent material; a first reflecting surface comprising an aminoalkyl moiety; and a second reflecting surface separated from said first reflecting surface by a distance, “d.”

These and other objects and features of the present invention will become more fully apparent when the following detailed description of the invention is read in conjunction with the accompanying drawings.

### BRIEF DESCRIPTION OF THE SEVERAL VIEWS OF THE DRAWING

FIG. 1 shows an optical assembly formed accordance to one embodiment of the invention;

FIG. 2 shows an optical assembly constructed according to another embodiment of the invention;

FIG. 3 shows a disposable multi-analyte optical assembly having an analyte-binding array and constructed according to another embodiment of the invention;

FIG. 4 illustrates initial steps involved in creating an amine-reactive biosensor according to one embodiment of the invention;

FIG. 5 illustrates exemplary conditions for carrying out initial steps for creating an amine-reactive biosensor for dry storage.

FIG. 6 illustrates subsequent steps involved in creating an amine-reactive biosensor according to one embodiment of the invention;

FIG. 7 shows an exemplary arrangement of reagents in a multi-well plate for making and using a plurality of amine-reactive biosensors in accordance with an embodiment of the invention;

FIG. 8 shows data obtained from a plurality of amine-reactive biosensors in accordance with an embodiment of the invention; and

FIG. 9 shows data from an experiment comparing the relatively stability of BSA on an optical element to desorption in the presence of 2% sodium dodecyl sulfate. BSA adsorbed and covalently linked using EDC/NHS is stable to desorption, while BSA that is adsorbed but not covalently linked desorbs in the presence of EDC/NHS.

### DETAILED DESCRIPTION OF THE INVENTION

### Definitions

Terms used in the claims and specification are to be construed in accordance with their usual meaning as understood by one skilled in the art except and as defined as set forth below. Numeric ranges recited in the claims and specification are to be construed as including the limits bounding the recited ranges.

An “analyte-binding molecule” refers to any molecule capable of participating in a specific binding reaction with an analyte molecule. Examples include but are not limited to, e.g., antibody-antigen binding reactions, drug-receptor binding interactions, and nucleic acid hybridization reactions.

A “specific binding reaction” refers to a binding reaction that is saturable, usually reversible, and that can be competed with an excess of one of the reactants. Specific binding reactions are characterized by complementarity of shape, charge, and other binding determinants as between the participants in the specific binding reaction.

An “antibody” refers to an immunoglobulin molecule having two heavy chains and two light chains prepared by any method known in the art or later developed and includes polyclonal antibodies such as those produced by inoculating a mammal such as a goat, mouse, rabbit, etc. with an immunogen, as well as monoclonal antibodies produced using the well-known Kohler Milstein hybridoma fusion technique. The term includes antibodies produced using genetic engineering methods such as those employing, e.g., SCID mice reconstituted with human immunoglobulin genes, as well as antibodies that have been humanized using art-known resurfacing techniques.

An “antibody fragment” refers to a fragment of an antibody molecule produced by chemical cleavage or genetic engineering techniques, as well as to single chain variable fragments (SCFvs) such as those produced using combinatorial genetic libraries and phage display technologies. Antibody fragments used in accordance with the present invention usually retain the ability to bind their cognate antigen and so include variable sequences and antigen combining sites.

A “small molecule” refers to an organic compound having a molecular weight less than about 500 daltons. Small molecules are useful starting materials for screening to identify drug lead compounds that then can be optimized through traditional medicinal chemistry, structure activity relationship studies to create new drugs. Small molecule drug compounds have the benefit of usually being orally bioavailable. Examples of small molecules include compounds listed in the following databases the contents of which are available online: MDL/ACD, MDL/MDDR, SPECS, the China Natural Product Database (CNPD), and the compound sample database of the National Center for Drug Screening.

A “water-soluble carbodiimide” refers to a chemical with a reactive carbodiimide group and that is sufficiently soluble in aqueous solution to couple a carboxyl group to a primary amine in an aqueous medium. Representative “water-soluble carbodiimides” include, but are not limited to, e.g., EDC, N,N′-dicyclohexylcarbodiimide, and N,N′-diisopropylcarbodiimide.

An “N-hydroxysuccinimide” refers to N-hydroxysuccinimide and analogs thereof, including but not limited to, e.g., Sulfo-NHS, and 2,3-dihydroxy-succinamide, that can be used with a water-soluble carbodiimide to increase the efficiency of a coupling reaction.

An “aminoalkyl moiety” refers to a molecule that includes a free amine group bonded to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl(allyl), cycloprop-1-en-1-yl; cycloprop-2-en-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like hydrocarbon group. The term is intended to cover hydrocarbon chains of any length, but preferably from C1 to C20, more preferably C1 to C10, and most preferably from C1 to C3, and is intended to cover straight and branched alkane, alkene, and alkyne groups. In the practice of the present invention, aminoalkyl moieties usually are functional groups added to the surface of an optical element using well-known chemistry, such as by treating a glass surface with an aminoalkylsilane to functionalize a glass or plastic surface with a free amine group. Representative aminoalkylsilanes include but are not limited to, e.g., 3-(Trimethoxysilyl)propylamine, and (3-Aminopropyl)tris[2-(2-methoxyethoxy)ethoxy]silane.

In the present specification, the phrase “quenching a reaction mixture” means chemically stabilizing a reactive intermediate by addition of a chemical that reacts with the reactive intermediate to form a stable adduct.

A “chaotrope” is a chemical agent that disrupts protein structure. Chaotropes include, but are not limited to detergents, urea, guanidine hydrochloride, salts, acids, and bases.

Abbreviations used in this application include the following: “PBS” refers to phosphate buffered saline (0.01 M phosphate buffer, 0.0027 M potassium chloride and 0.137 M sodium chloride, pH 7.4); “PBST” refers to PBS+0.02% (v/v) Tween 20; “NHS” refers to N-hydroxysuccinimide; “MW” refers to molecular weight; “Sulfo-SMCC” refers to sulfosuccinimidyl 4-M-maleimidomethyl]cyclohexane-1-carboxylate; “EDC” refers to (1-Ethyl-3-[3-dimethylaminopropyl]carbodiimide) and salts thereof.

It must be noted that, as used in the specification and the appended claims, the singular forms “a,” “an,” and “the” include plural referents unless the context clearly dictates otherwise.

### Advantages and Utility

The advantages and utility of the invention are illustrated by reference to the Figures and Examples as described in greater detail below. These include the ability to covalently couple a layer of analyte-binding molecules to a polycarboxylic acid-containing polymer that has been coupled to a reflecting surface. Biosensors that include a layer of covalently coupled analyte-binding molecules in accordance with the present invention are extremely stable, and so are capable of being regenerated after use. In addition, non-specific binding can be reduced, and native binding activity of a biomolecular interaction can be improved through the use of a macromolecular spacer that distances the biomolecular interaction from the sensor surface. Another advantage provided by certain embodiments of the invention is the ability to couple an analyte-binding molecule to an optical element using methods that conserve a sample of the analyte-binding molecule by preventing the sample from being diluted with buffers or lost to dead volume. The present invention may be practiced in a multiplexed array embodiment, further speeding the analysis of biomolecular binding reactions.

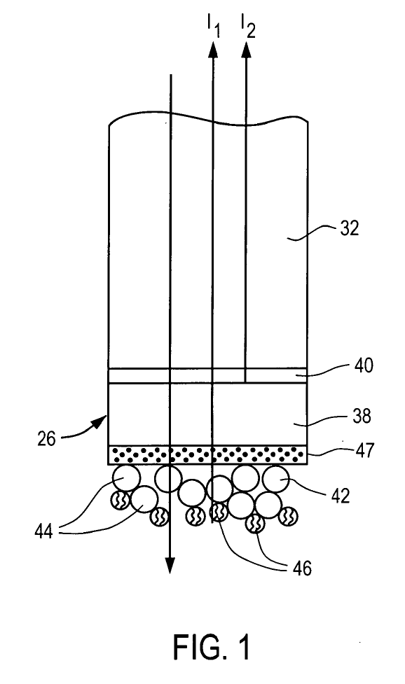

FIG. 1 shows, in schematic view, an optical assembly 26 adapted for coupling to a light source via optical waveguide or fiber 32. When used as a biosensor in a fiber-optic assay apparatus based on phase-shift interferometry (as illustrated FIG. 1 of the '062 application), an interference pattern is generated by rays I₁ and I₂. Changes in that interference pattern are used to detect binding of analytes 46 to analyte-binding molecules 44.

Suitable optical fiber and coupling components are detailed in the above-cited '453 patent. One exemplary coupler is commercially available from many vendors including Ocean Optics (Dunedin, Fla.).

In the embodiment shown in FIG. 1, the optical assembly 26 is fixedly attached to an adjoining portion of the distal end region of an optical fiber 32, although disposable fiber optic tips as described in co-owned U.S. patent application Ser. No. 11/423,671 (which is hereby incorporated by reference in its entirety for all purposes) are expressly contemplated to be within the scope of the present invention. As shown, the assembly 26 includes a transparent optical element 38 having first and second reflecting surfaces 42, 40 formed on its lower (distal) and upper (proximal) end faces, respectively. In one embodiment of the invention, the thickness “d” of the optical element between its distal and proximal surfaces, i.e., between the two reflecting surfaces, is at least 50 nm, and preferably at least 100 nm. An exemplary thickness is between about 100-5,000 nm, preferably 400-1,000 nm. The first reflecting surface 42 comprises a polycarboxylated polymer 47 that, following the derivatization methods of the present invention, forms amide bonds between the surface chemistry and the polymer and between the polymer and a layer of analyte-binding molecules, such as molecules 44, which are capable of binding analyte molecules 46 specifically and with high affinity. That is, the analyte and anti-analyte molecules are opposite members of a binding pair of the type described above, which can include, without limitations, antigen-antibody pairs, complementary nucleic acids, and receptor-binding agent pairs.

The index of refraction of the optical element is preferably similar to that of the first reflecting surface, so that reflection from the lower distal end of the end optical assembly occurs predominantly from the layer that includes the polycarboxylated polymer and the analyte-binding molecules, rather than from the interface between the optical element and the layer that includes the polycarboxylated polymer and the analyte-binding molecules. Similarly, as analyte molecules bind to the lower layer of the optical assembly, light reflection form the lower end of the assembly occurs predominantly from the layer formed by the analyte-binding molecules and bound analyte, rather than from the interface region. One exemplary material forming the optical element is SiO₂, e.g., a high-quality glass having an index of refraction of about 1.4-1.5. The optical element can also be formed of a transparent polymer, such as polystyrene or polyethylene, having an index of refraction preferably in the 1.3-1.8 range.

The second reflecting surface in the optical assembly is formed as a layer of transparent material having an index of refraction that is substantially higher than that of the optical element, such that this layer functions to reflect a portion of the light directed onto the optical assembly. Preferably, the second layer has a refractive index greater than 1.8. One exemplary material for the second layer is Ta₂O₅ with refractive index equal to 2.1. The layer is typically formed on the optical element by a conventional vapor deposition coating or layering process, to a layer thickness of less than 50 nm, typically between 5 and 30 nm.

The thickness of the first (analyte-binding) layer is designed to optimize the overall sensitivity based on specific hardware and optical components. The present invention uses conventional immobilization chemistries to covalently attach a polycarboxylated polymer to the lower surface of the optical element and in turn to covalently attach an analyte-binding molecule to the polycarboxylated polymer. The present invention contemplates using amide bonds to make these covalent attachments. For example, a variety of bifunctional reagents containing a siloxane group for chemical attachment to SiO₂, and an amine group, such as, e.g., 3-(Trimethoxysilyl)propylamine, and (3-Aminopropyl)tris[2-(2-methoxyethoxy)ethoxy]silane can be used for functionalizing a glass surface with a free amine group. As described in this disclosure, a water soluble carbodiimide along with an N-hydroxysuccinimide then can be used for attaching a polycarboxylated polymer such as a polypeptide (e.g., BSA, casein, or another inert protein that will not interfere with the specific binding of analyte to the analyte-binding molecule) or a carboxy terminated dendrimer such as, e.g., NH₂(CH₂)₂NH₂]:(G=3.5);dendri PAMAM(NHCH₂CH₂COONa)₆₄, to a surface of an optical element. The analyte-binding molecule can be any type of molecule comprising a free amine that can be covalently attached via an amide bond to the polycarboxylated polymer, including, by way of example, but not limitation, a glycoprotein, a peptide, a nucleic acid, a co-factor, a small molecule, a cell-surface protein (isolated or present in a cell surface membrane), or a viral coat protein (isolated or present in the viral capsid).

The polycarboxylated polymer/analyte-binding layer is preferably formed under conditions in which the distal surface of the optical element is densely coated, so that binding of analyte molecules to the layer forces a change in the thickness of the layer, rather than filling in the layer. The polycarboxylated polymer/analyte-binding layer can be either a monolayer or a multi-layer matrix.

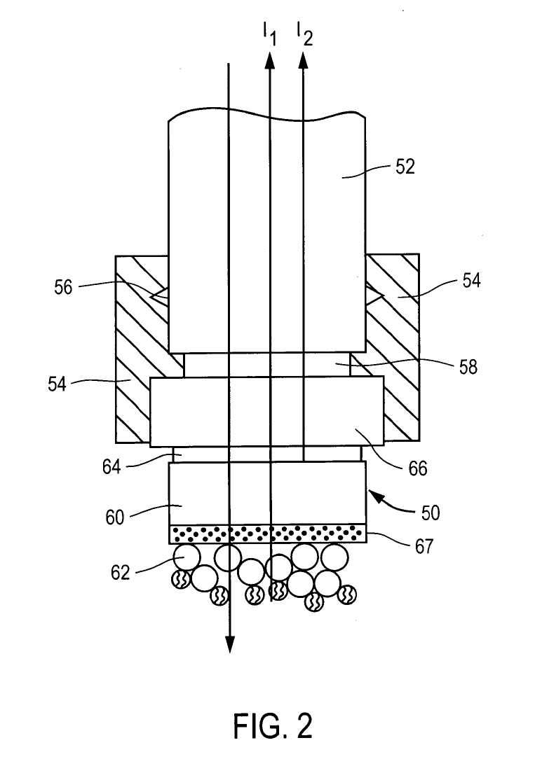

FIG. 2 shows an optical assembly 50 that is removably carried on the distal end of an optical fiber 52 in an assay apparatus. The optical element includes a plurality of flexible gripping arms, such as arms 54, that are designed to slide over the end of the fiber and grip the fiber by engagement of an annular rim or detente 56 on the fiber with complementary-shaped recesses formed in the arms, as shown. This attachment serves to position the optical assembly on the fiber to provide an air gap 58 between the distal end of the fiber and the confronting (upper) face of the assembly, of less than 100 nm or greater than 2 μm. With an air gap of greater than about 100 nm, but less that 2 μm, internal reflection from the upper surface of the optical assembly can contribute significantly to undesirable fringes that can adversely impact the detection accuracy.

With continued reference to FIG. 2, the optical assembly includes a first optical element 60 similar to optical element 38 described above, and having first and second reflective layers 62, 64, respectively, corresponding to above-described reflective layers 42, 40, respectively. Reflective layer 62 comprises a polycarboxylated polymer 67 that, following the derivatization methods of the present invention, forms amide bonds between the surface chemistry at the distal surface of element 60 and the polymer and between the polymer and the analyte-binding molecule. The assembly further includes a second optical element 66 whose thickness is preferably greater than 100 nm, typically at least 200 nm, and whose index of refraction is similar to that of first optical element 60. Preferably, the two optical elements are constructed of the same glass or a polymeric material having an index of refraction of between about 1.4 and 1.6. Layer 64, which is formed of a high index of refraction material, and has a thickness preferably less than about 30 nm, is sandwiched between the 2 optical elements as shown.

In operation, the optical assembly is placed over the distal fiber end and snapped into place on the fiber. The lower surface of the assembly is then exposed to a sample of analyte, under conditions that favor binding of sample analyte to the analyte-binding molecules comprising reflective layer 62. As analyte molecules bind to this layer, the thickness of the layer increases, increasing the distance “d” between reflective surfaces 62 and 64. This produces a shift in the extrema of the interference wave produced by reflection from the two layers. This shift in extrema or wavelength, or wavelength period, in turn, is used to determine the change in thickness at the lower (distal-most) reflecting layer. After use, the optical assembly can be removed and discarded, and replaced with fresh element for a new assay, for assaying the same or a different analyte.

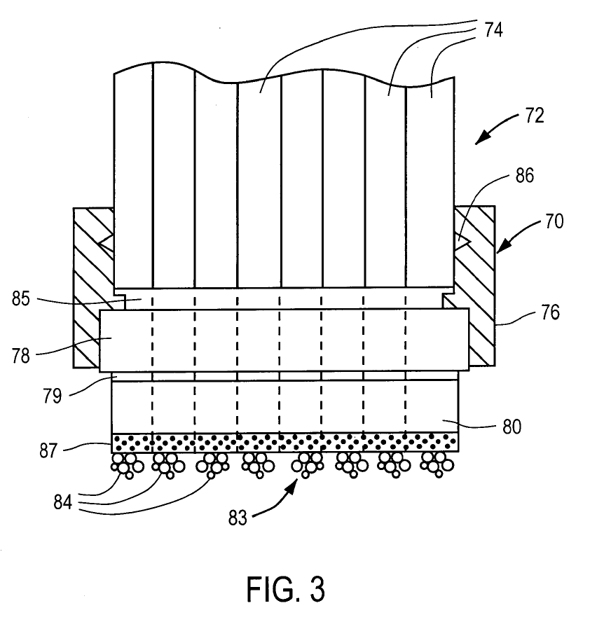

FIG. 3 illustrates an optical assembly and fiber bundle in an embodiment of the invention designed for detecting one or more of a plurality of analytes, e.g., different-sequence nucleic acid analytes, in a sample. A fiber bundle 72 is composed of an array, e.g., a circular array, for individual optical fibers, such as fibers 74. The optical assembly, indicated generally at 70, is composed of the basic optical elements described above with reference to FIG. 2, but in an array format. Specifically, a first optical element 80 in the element provides at its lower distal surface, an array of analyte-reaction regions, such as regions 84, each containing a layer of analyte-binding molecules effective to bind to one of the different analytes in the sample. Each region 84 also includes a polycarboxylated polymer 87 that, following the derivatization methods of the present invention, forms amide bonds between the surface chemistry and the polymer and between the polymer and the analyte-binding molecule. Each region forms a first reflective layer in the optical assembly. One preferred sensing provides an array of different-sequence nucleic acids, e.g., cDNAs or oligonucleotides, designed to hybridize specifically with different-sequence nucleic acid analyte species in a sample. That is, the array surface forms a “gene chip” for detecting each of a plurality of different gene sequences.

Also included in the optical assembly are a second optical element 78 and a layer 79 of high index of refraction material sandwiched between the two optical elements, and which provides the second reflecting surface in the optical assembly. The assembly is carried on the fiber bundle 72 by engagement between a pair of flexible support arm, such as arm 76 and an annular rim or detente 86 on the bundle. With the assembly placed on the fiber bundle, the lower distal ends of the fibers are spaced from the confronting surface of optical element 78 by an air gap 85 whose spacing is preferably less than 100 nm or greater than 2 μm. Further, each of the fibers is aligned with a corresponding assay region of the optical assembly, so that each fiber is directing light on, and receiving reflected light from, its aligned detection region. Similarly, the optical coupler in the apparatus, which serves to couple multiple fibers to the detector, preserves the alignment between the array regions and corresponding positions on an optical detector, e.g., a two-dimensional CCD. The materials and thickness dimensions of the various optical-assembly components are similar to those described above with respect to FIG. 2.

### Kits of the Invention

In addition to the methods and articles of manufacture disclosed in the present specification, the invention also includes kits for derivatizing optical assemblies with a layer of analyte-binding molecules. The kits preferably comprise a polymer comprising a plurality of carboxy groups (i.e., a polycarboxylated polymer), a water-soluble carbodiimide, an N-hydroxysuccinimide, a quenching reagent, instructions for use, and packaging. In one embodiment, the kit includes EDC as the water-soluble carbodiimide. In another embodiment, the kit includes N-hydroxysuccinimide as the N-hydroxysuccinimide. In yet another embodiment, the kit includes ethanolamine as the quenching reagent. In still other embodiments, the kit includes dry salts for diluting with water to make buffers used with the kit. In another embodiment, the kit includes containers. In some embodiments, the containers are multi-well plates. In other embodiments, the kits include an optical assembly comprising an optical element adapted for coupling to a light source via a fiber. In preferred embodiments, the optical assembly includes an optical element that has a functionalized surface. Especially preferred functionalized surfaces are APS (aminopropylsilane) functionalized surfaces. In other preferred embodiments, the instructions for use include instructions for carrying out the protocols substantially as described in Examples 4 and/or 5.

### Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### Example 1

### Preparing an Aminopropylsilane Coated Tip

A two-layer configuration on the tip of an optic fiber in accordance with the embodiment illustrated in FIG. 1 was constructed. The thickness of the first Ta₂O₅ layer is 25 nm and the thickness of the second SiO₂ layer is 770 nm. The fiber was purchased from Ocean Optics (Dunedin, Fla.). It was manually cut into segments that are 40 mm long. Both ends of these segments were polished to standard mirror surface quality. The polishing method used here was exactly the same as those for optical lenses and mirrors. One surface of these fiber segments was outsourced to an optical coating house for Ta₂O₅ layer and SiO₂ layer. This vendor employed an ion-beam assisted physical vapor deposition (IAPVD) coater made by Leybold. IAPVD is a commonly used coating technique for anti-reflection and optical filters. The experimental steps included the following (all steps are performed at room temperature unless otherwise noted):

Aminopropyl coated tips were prepared using the following procedure: Coated optical fibers were cleaned using oxygen plasma in a Plasma Cleaning System (Model G-1000; Yield Engineering Systems Inc., San Jose, Calif.) for 5 minutes using a 200 watt setting. The oxygen plasma-cleaned fibers were transferred to a Silane Vapor Deposition System (Model YES-1224; Yield Engineering Systems Inc., San Jose, Calif.). The chamber was warmed up for 20 Minutes (Process temperature 155° C.; pressure 0.75 torr), then chemical vapor deposition of APS using a 97% solution of 3-(Trimethoxysilyl)propylamine according to the manufacturer's instructions was performed for 5 min at 155° C.; pressure 13 torr.

### Example 2

### Adsorbing a Polycarboxylated Polymer to the Aminopropylsilane Coated Tip

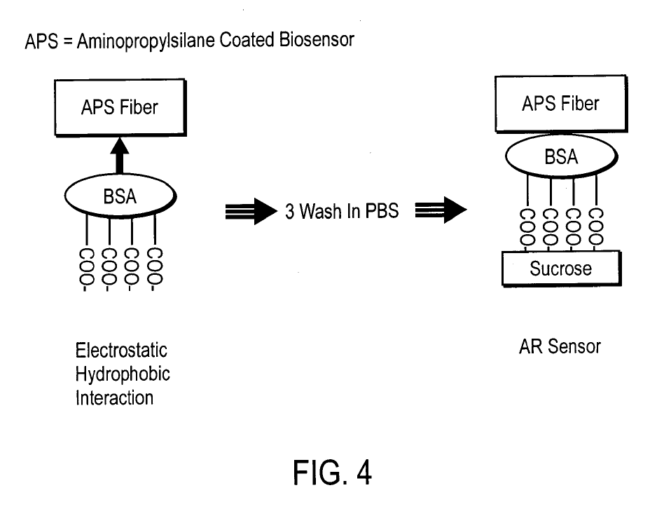

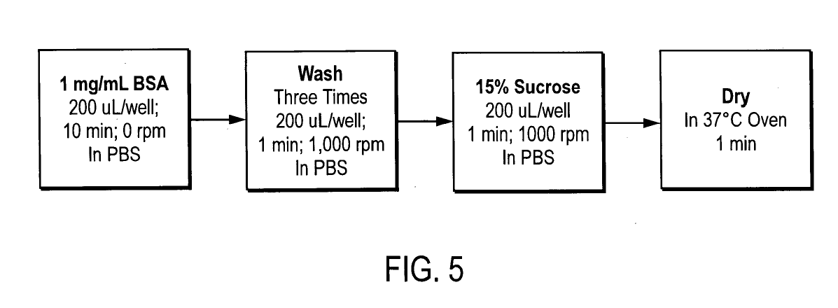

In the initial steps for preparing an amine-reactive biosensor tip, an aminopropylsilane coated tip as prepared in Example 1 was coated with BSA, washed in PBS, and then stabilized with a sucrose coating, which allows for dry storage of the tip. The general scheme is illustrated in FIG. 4. As noted in FIG. 4, this results in a tip that has the polycarboxylated polymer (in this Example, the polymer is BSA) adsorbed through non-covalent interactions, and allows manufacturing to proceed up to this intermediate step. The tip is subsequently stored in dry form until it is covalently derivatized with an analyte-binding molecule. Further details for these steps are provided in FIG. 5. In this Example, the process steps are carried out in black, flat-bottom 96 well plates (available from Grener Bio-One, catalog number 655209). To coat the APS tip with BSA, the tip is immersed in 200 μL of a 1 mg/mL solution of BSA dissolved in PBS. The tip is immersed for 10 minutes at room temperature with no agitation of the tip of BSA solution.

Next, the tip is washed by three successive washes in 200 μL of PBS at room temperature. The plate is agitated at 1,000 rpm.

Stabilization is achieved by immersing the tips into 200 μL, of a 15% (w/v) solution of sucrose dissolved in PBS and held at room temperature. The plate again is agitated at 1,000 rpm. Following the sucrose immersion, the tip is dried by moving it to an oven set to 37° C. for 1 minute.

### Example 3

### Covalent Attachment of the Polycarboxylated Polymer and the Analyte-Binding Molecule to the Amine-Reactive Biosensor

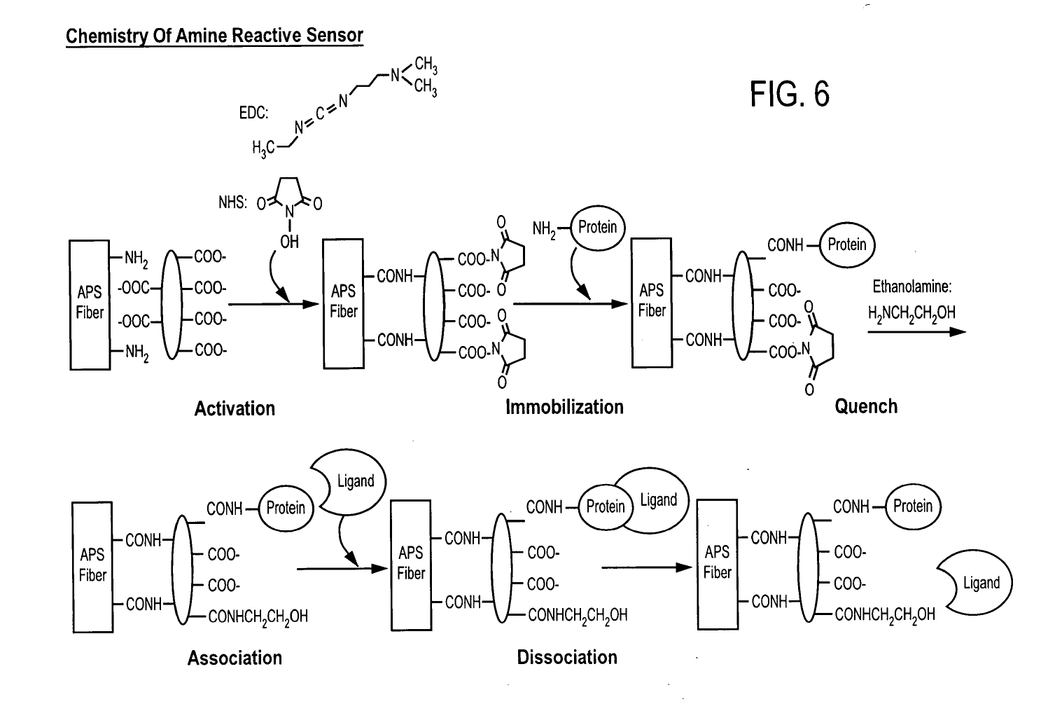

The amine-reactive biosensor of Example 2 was further processed to covalently attach both the BSA and an analyte-binding molecule (in this Example, several proteins were used as exemplary analyte-binding molecules protein). The general chemistry for this exemplified embodiment is illustrated in FIG. 6. The top panels illustrate the APS coated fiber (rectangle labeled “APS fiber”) to which the polycarboxylated polymer (ellipse showing four carboxyl groups) has been adsorbed. The carboxyl groups on the BSA are activated in an activation step by exposing the tip to a solution of EDC and NHS. Once the carboxyl groups are activated, the tip is brought into contact with a solution of an analyte-binding molecule (protein in this Example) in an immobilization step. A free amine in the analyte-binding molecule forms an amide bond with an activated carboxyl group on the polycarboxylated polymer. Another amide bond is formed between an activated carboxyl group on the polycarboxylated polymer and an amine group on the surface of the glass fiber. Once immobilization is completed, the reaction is quenched. In this Example, ethanolamine is used as a quenching agent.

The bottom panels of FIG. 6 illustrate using the completed amine-reactive sensor to characterize association and dissociation reactions between the analyte-binding molecule and a ligand to which it specifically binds. The covalent attachment of the analyte-binding molecule to the biosensor (through the polycarboxylated polymer) makes the sensor extremely stable, allowing slow dissociation reactions to be accurately studied, and permitting regeneration of the sensor by stripping the ligand from the analyte-binding molecule using a chaotrope.

Further details for the activation, immobilization and quench steps follow below.

Materials:

EDC, Sigma (catalog number E7750);

NHS, Aldrich (catalog number 130672)

Immobilization Buffer (100 mM MES, pH 5 suggested starting point—one or ordinary skill having the benefit of this disclosure will readily understand how to adjust the pH of the immobilization buffer to optimize coupling.)

Quench Buffer, 1M Ethanolamine, pH 8.5

Sensor Storage Buffer (buffer into which ligand is to be diluted), 1× PBS recommended

Introduction:

The amine-reactive biosensor allows for the coupling of an analyte-binding molecule such as, e.g., a protein, to the biosensor surface via accessible amine groups. The coupling procedure is a simple three step protocol based on well known amide bond formation chemistry.

Preparation and Storage of Coupling and Quench Reagents:

A 0.4M EDC solution is prepared by dissolving 3.1 g EDC in 100 mM MES buffer (pH 5.0) to a final volume 40 mL. For storage, 1 mL aliquots of the EDC solution are dispensed into 2 mL eppendorf tubes and are stored at −20° C. Aliquots are stable for up to 3 months.

A 0.1M NHS solution is prepared by dissolving 0.46 g NHS in pH 5.0, 100 mM MES buffer to a final volume of 40 mL. For storage, 1 mL aliquots of the NHS solution are dispensed into 2 mL eppendorf tubes and are stored at −20° C. Aliquots are stable for up to 3 months.

Quench buffer is prepared by dissolving 48.8 g ethanolamine in water to a final volume of 500 mL. The pH is adjust pH to 8.5 using KOH, and the solution is stored at room temperature.

### Example 4

### Online Immobilization and Assay Protocol

This Example provides a typical protocol for online immobilization and ligand binding using FortéBio's Octet system and the associated Octet software, both available from FortéBio, Inc., 1360 Willow Road, Suite 205, Menlo Park, Calif. 94025. This protocol is used as a general guideline and may require further optimization for specific applications. One of ordinary skill having the benefit of this disclosure will readily understand how to adjust the protocol by varying, e.g., the concentration and composition of reagents, the ionic strength, and the pH to obtain robust immobilization and signal to noise ratios during the assays. The exemplified protocol uses a protein as an analyte-binding molecule, but the invention encompasses any analyte-binding molecule having a free amine group that can be coupled to the amine-reactive biosensor using the methods disclosed herein.

An appropriate amount and concentration of protein to be immobilized (2 mL of solution is sufficient for 8 sensors) is prepared. The protein ideally is in a solution of low ionic strength (˜100 mM) and at a pH below its isoelectric point. If the optimal immobilization conditions are not known, a solution of 25 μg/mL of protein in 100 mM MES (pH 5.0) is recommended as a starting condition. By changing the protein concentration and buffer pH the immobilization can be further optimized if needed.

An appropriate amount and concentration of the ligand to be tested is prepared using sensor storage buffer. As a general guideline, 2 mL of 40 nM ligand is sufficient for use with 8 sensors.

The EDC and NHS solutions are thawed and mixed 1:1. 2 mL total mixed solution (i.e., 1 mL of EDC and 1 mL NHS) is a sufficient amount of mixture for coupling 8 sensors.

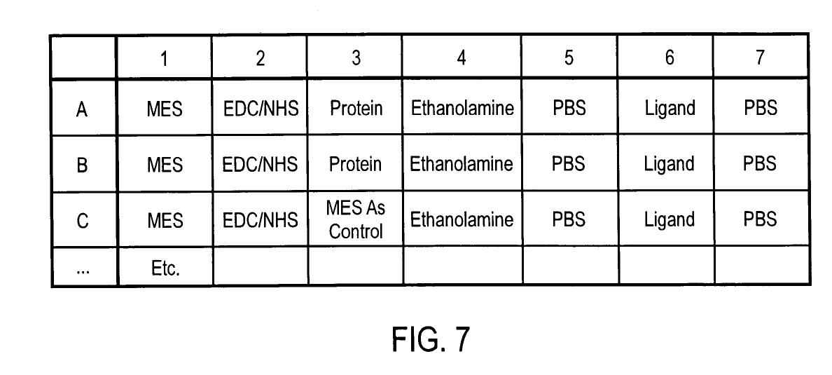

200 μL of the appropriate solutions are dispensed into each column of a sample plate (black flat bottom 96 well plate) according to the method file setup. A representative plate setup is illustrated in FIG. 7. Each row (A, B, C, etc.) illustrates an independent activation/immobilization/quench/assay combination. An amine-reactive biosensor tip, such as the tip described in Example 2 is used. That tip includes a layer of BSA adsorbed to the tip of an aminopropylsilane coated glass fiber that is coated with a sucrose solution and dried. Moving the tip from well to well in a left to right sequence across a row executes steps that include activation of the sensor, immobilization of the protein, quench of unreacted activated sites, equilibration, binding and dissociation of the ligand.

Setup is completed by assigning the file name and sub-directory for the saved data.

The sensors are pre-wetted in the immobilization buffer using a flat bottom 96 well plate and FortéBio sensor tray. Both the sensor tray and the sample plate are placed into the Octet.

The “Start-delay” is set for 300 seconds by checking the dialogue box. This allow the solutions in the sample plate to warm to 30° C. The “shaking the plate while waiting to start” option is selected.

The run is started by clicking “Go.”

### Example 5

### Off-Line Batch Immobilization Protocol

This Example provides a typical protocol for off-line immobilization using FortéBio's Octet system. This protocol is useful as a general guideline for preparing 8 sensors, and can be readily scaled up for specific applications.

Reagents and sample preparations are same as steps described beginning in Example 3 and proceeding to the EDC and NHS thaw/mix step of Example 4.

200 μL of Immobilization Buffer is dispensed into column 1 of a black flat-bottom micro titer plate.

200 μL of EDC/NHS mixture is dispensed into column 2 of the black flat-bottom micro titer plate.

200 μL of an analyte-binding molecule solution (for the analyte-binding molecule to be immobilized) is dispensed into column 3 of the black flat-bottom micro titer plate.

200 μL of ethanolamine is dispensed into column 4 of the black flat-bottom micro titer plate.

200 μL of Sensor Storage Buffer is dispensed into column 5 of the black flat-bottom micro titer plate.

Eight sensors are placed sensors in the column 1 (MES-containing) wells and are incubated for 5 minutes.

The sensors are moved to the column 2 (EDC/NHS-containing) wells and are incubated for 5 minutes.

The sensors next are moved into the column 3 (analyte-binding molecule-containing) wells. They are incubated for three-times the online immobilization time for the same analyte-binding molecule.

Next the sensors are moved into the column 4 (ethanolamine-containing) wells and are incubated for 5 minutes.

The sensors then are moved into the column 5 (Sensor Storage Buffer-containing) wells and are stored at 4° C. with lid in place until use.

The sensors should be equilibrated at room temperature for 15 minutes prior to use.

For long term storage (>3 days), the sensors are dipped into a 15% (w/v) sucrose solution (made in Sensor Storage Buffer) for 1 minute and air-dried for 30 minutes. Dried sensors are stored in a pouch containing a dessicant.

### Example 6

### Multiplexed Binding and Dissociation Studies Using Amine-Reactive Biosensors

Amine-reactive biosensors were prepared and used according to the online immobilization and assay protocol described in Example 4. Four different analyte-binding molecule/analyte pairs were tested along with three controls. The on-line protocol was used with the following solutions, concentrations and reaction times:

| | EDC/NHS | IMMOBILIZATION | QUENCH | BASELINE | BINDING | DISSOCIATION |
|---|---|---|---|---|---|---|
| sensor | (300 seconds) | (1400 seconds) | (300 seconds) | (300 seconds) | (600 seconds) | (1000 sconds) |
| A | 0.2 M EDC | antigen 1 (65 KD) | 1M | PBS | antibody 1 (150 KD) | PBST |
| | 0.05M NHS | 25 ug/mL in MES pH 5 | ethanolamine | | 40 nM in PBST | |
| B | 0.2 M EDC | antigen 2 (40 KD) | 1M | PBST | antibody 2 (150 KD) | PBST |
| | 0.05M NHS | 25 ug/mL in MES pH 5 | ethanolamine | | 40 nM in PBST | |
| C | 0.2 M EDC | MES pH 5 | 1M | PBST | antibody 1 (150 KD) | PBST |
| | 0.05M NHS | | ethanolamine | | 40 nM in PBST | |
| D | 0.2 M EDC | MES pH 5 | 1M | PBST | antibody 2 (150 KD) | PBST |
| | 0.05M NHS | | ethanolamine | | 40 nM in PBST | |
| E | 0.2 M EDC | antibody 1 (150 KD) | 1M | PBST | antigen 3 (30 KD) | PBST |
| | 0.05M NHS | 25 ug/mL in MES pH 5 | ethanolamine | | 40 nM in PBST | |
| F | 0.2 M EDC | MES pH 5 | 1M | PBST | antigen 1 (65 KD) | PBST |
| | 0.05M NHS | | ethanolamine | | 40 nM in PBST | |
| G | 0.2 M EDC | antibody 1 (150 KD) | 1M | PBST | antigen 1 (65 KD) | PBST |
| | 0.05M NHS | 25 ug/mL in MES pH 5 | ethanolamine | | 40 nM in PBST | |

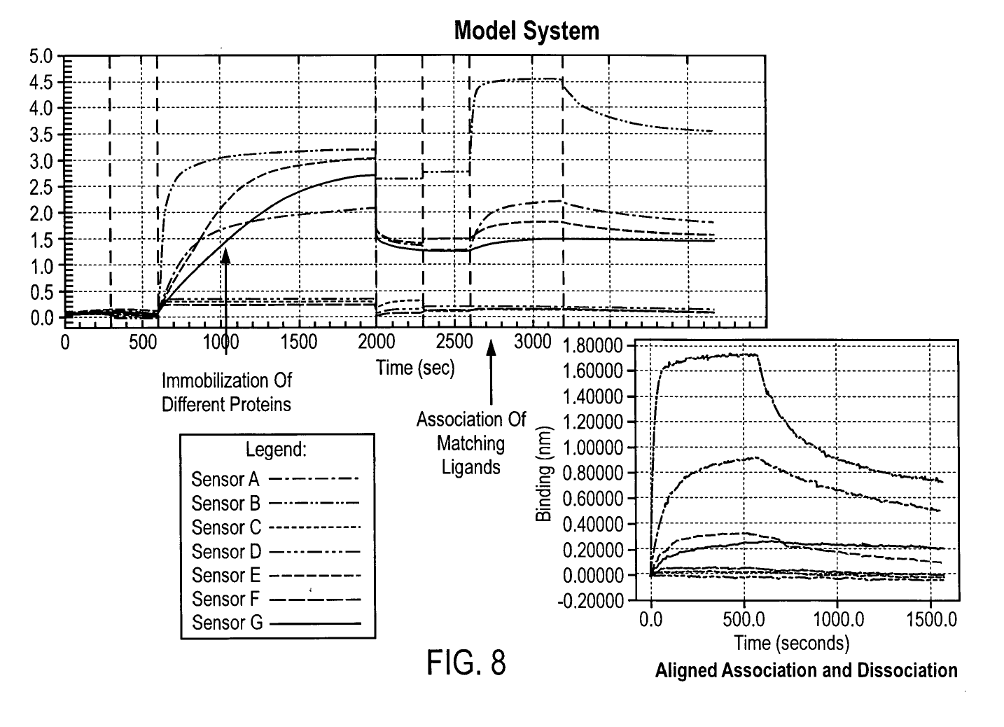

The results are illustrated in FIG. 8. The set of traces in the top panel illustrates output of the Octet system as sensors are moved from column 1 through column 7, corresponding to the well solutions illustrated in FIG. 7. The vertical dashed lines appear at the transition points between wells. Note that the traces remain essentially flat for the first two incubations (MES and EDC/NHS). Once the tips are moved into protein-containing solutions (top four traces), the traces ascend as the proteins become immobilized on the sensor tip. Notice that the kinetics of immobilization and the depth of the immobilized protein layer vary as a function of protein. The thickness of the of immobilized protein layer is a function of the immobilization density and the molecular size for the protein. This thickness can be controlled by altering the protein concentration in the immobilization solution, the pH of the immobilization buffer and the immobilization time. The bottom three traces are controls in which no protein was present in the immobilization solution. Note that the control traces remain essentially flat.

Once the immobilization step is completed, the tips are moved to quench buffer, and then subsequently are washed in PBS. Following the PBS wash, the tips are moved to wells containing the cognate ligands. Again, note that the top four traces rise, as ligand binds to the analyte-binding molecules. The control traces remain essentially flat. Finally, the sensors are moved to PBS to monitor dissociation of ligand from the analyte-binding molecule. The inset at the bottom part of FIG. 8 illustrates aligned association and dissociation traces.

### Example 7

### Multiplexed Binding and Dissociation Studies Using Amine-Reactive Biosensors

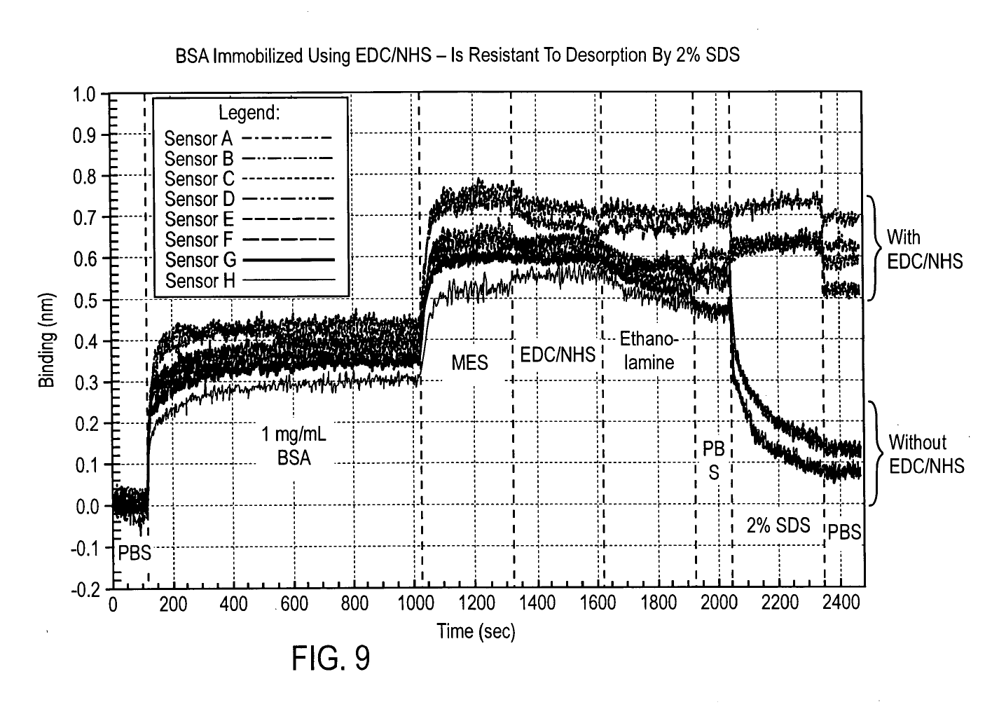

This Example illustrates that BSA immobilized onto an APS sensor tip resists desorption by the detergent chaotrope, sodium dodecyl sulfate (SDS). A total of seven APS sensors were used in a protocol that compared the stability of BSA coupled to the tip using the EDC/NHS protocol as compared to BSA that was adsorbed to the tip, but not covalently linked. Results are shown in FIG. 9. The figure shows the output of a Fortébio Octet instrument as tips are moved from solution to solution. Note that as the tips are moved from PBS into 1 mg/mL BSA, the traces rise as BSA adsorbs onto the APS glass fiber tip. The tips next are moved to MES immobilization buffer and following this step, four of the seven tips are moved to EDC/NHS, and three are moved to a control solution that lacks EDC/NHS. The tips next are moved into ethanolamine (quench). The quenching step is followed by incubation in a 2% (w/v) SDS solution in PBS. The tips that were incubated in EDC/NHS resist desorption of the BSA in the presence of SDS, while those that were not incubated in EDC/NHS show rapid desorption of the BSA from the APS tip.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A method for derivatizing an optical assembly, comprising:
providing said optical assembly, said assembly comprising an optical element adapted for coupling to a light source via a fiber, said optical element comprising a transparent material, a first reflective surface comprising an aminoalkyl moiety, and a second reflecting surface separated from said first surface by a distance, d;
a polymer comprising plurality of carboxyl groups; and
an analyte-binding molecule comprising an amine group;
activating said plurality of carboxyl groups by exposing said polymer to a water-soluble carbodiimide and an N-hydroxysuccinimide;
exposing said plurality of activated carboxyl groups to a reaction mixture comprising said analyte-binding molecule to form a first amide bond between said polymer and said aminoalkyl moiety and a second amide bond between said polymer and said analyte-binding molecule; and
quenching said reaction mixture, thereby derivatizing said optical assembly.

2. The method of claim 1, wherein said polymer is a polypeptide.

3. The method of claim 2, wherein said polypeptide is bovine serum albumin.

4. The method of claim 1, wherein said analyte-binding molecule is a polypeptide.

5. The method of claim 1, wherein said analyte-binding molecule is a nucleic acid.

6. The method of claim 1, wherein said distance, d, is at least 50 nm.

7. The method of claim 1, wherein said water-soluble carbodiimide is EDC.

8. The method of claim 1, further comprising exposing the optical element to a solution comprising a sugar and subsequently drying the optical element.

9. The method of claim 8, wherein said sugar is sucrose.

10. A method for regenerating an optical assembly derivatized according to the method of 1, comprising exposing the derivatized assembly to a chaotrope, wherein said exposure is sufficient to remove a non-covalently bound material from said optical element.

11. The method of claim 10, wherein said chaotrope is selected from the group consisting of an acid, a base, a salt, a detergent, urea, and a guanidinium ion.

12. The method of claim 11, wherein the detergent is sodium dodecyl sulfate.

13. An optical assembly according to the method of claim 1.

14. An optical assembly according to the method of claim 8.

15. A kit for derivatizing an optical assembly with a layer of analyte-binding molecules comprising an amine group, comprising at least three of a polymer comprising a plurality of carboxyl groups; a water-soluble carbodiimide; an N-hydroxysuccinimide; a quencher; instructions for use; and packaging.

16. The kit of claim 15, wherein said water-soluble carbodiimide is EDC.

17. The kit of claim 15, wherein said polymer is a polypeptide.

18. The kit of claim 15, further comprising a sugar.

19. The kit of claim 15, further comprising a chaotrope.

20. The kit of claim 15, further comprising an optical assembly, said assembly comprising an optical element adapted for coupling to a light source via a fiber, said optical element comprising: a transparent material; a first reflecting surface comprising an aminoalkyl glass; and a second reflecting surface separated from said first reflecting surface by a distance, d.
